SEQUENCE LISTING (1 sequence)

SEQ ID NO: 1 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

(19) United States
(12) Patent Application Publication
(10) Pub. No.: US 2015/0231167 A1
(43) Pub. Date: Aug. 20, 2015

(54) TREATMENT OF HEMATOLOGICAL NEOPLASMS

(71) Applicant: THOMAS JEFFERSON UNIVERSITY, PHILADELPHIA, PA (US)

(72) Inventors: Marja Tuuli Nevalainen, Gladwyne, PA (US); Zhiyong Liao, Wynnewood, PA (US)

(21) Appl. No.: 14/421,623

(22) PCT Filed: May 10, 2013
(86) PCT No.: PCT/US13/40478

§ 371 Date: Feb. 13, 2015

Related U.S. Application Data
(60) Provisional application No. 61/683,910, filed on Aug. 16, 2012.

(51) Int. Cl. A61K 31/7076; A61K 45/06

(52) U.S. Cl. CPC A61K 31/7076; A61K 45/06

(57) ABSTRACT

Hematological neoplasms resistant to treatment with an ATP-competitive inhibitor of BCR-ABL are treated by administration of N⁶-benzyladenosine, N⁶-benzyladenosine-5′-monophosphate, or pharmaceutically acceptable salts thereof.

### CROSS-REFERENCE OF RELATED APPLICATION

The benefit of the filing date of U.S. Provisional Patent Application No. 61/683,910, filed Aug. 16, 2012, is hereby claimed. The entire disclosure of the aforesaid application is incorporated herein by reference.

### FIELD OF THE INVENTION

The invention relates to the treatment of prostate cancer and hematological neoplasms.

### SEQUENCE LISTING

The instant application contains a Sequence Listing which has been submitted in ASCII format via EFS-Web and is hereby incorporated by reference in its entirety. Said ASCII copy, created on May 8, 2013, is named 37075_{—}0290_{—}00_WO_SL.txt and is 10,045 bytes in size.

### BACKGROUND OF THE INVENTION

Hematological neoplasms, also known as hematological malignancies, comprise cancers that affect blood, bone marrow, and lymph nodes. They may be driven by chromosomal translocations. Hematological neoplasms may derive from myeloid or lymphoid cell lineages. Lymphomas, lymphocytic leukemias, and myeloma are of lymphoid origin. Acute and chronic myelogenous leukemia (also known and myeloid leukemia), myelodysplastic syndromes and myeloproliferative diseases are myeloid in origin. Hematological neoplasms include the following specific disorders, for example: Acute lymphoblastic leukemia (ALL), a cancer of the white blood cells characterized by excess lymphoblasts; acute myelogenous leukemia (AML), a cancer of the myeloid line of blood cells, characterized by the rapid growth of abnormal white blood cells; acute monocytic leukemia (AMoL, or AML-M5), which is considered a type of AML; chronic lymphoid leukemia (CLL), also known as B-cell chronic lymphocytic leukemia (B-CLL), a cancer that affects B cell lymphocytes; chronic myelogenous (or myeloid) leukemia (CML), also known as chronic granulocytic leukemia (CGL), a cancer of the white blood cell; Hodgkin's lymphoma; non-Hodgkin lymphomas (NHLs), which comprise a group of blood cancers that include any type of lymphoma except Hodgkin's lymphomas; and myelomas, such as multiple myeloma, which is a cancer of plasma cells.

CML is associated with a specific chromosomal abnormality, namely the Philadelphia or Ph¹ chromosome, which results from the translocation of the proto-oncogene c-abl from the long arm of chromosome 9 to the breakpoint cluster region (bcr) on chromosome 22, resulting in the formation of bcr-abl hybrid genes. The c-abl proto-oncogene normally encodes a protein (ABL) having tyrosine kinase activity. In cells carrying bcr-abl hybrid genes, the oncogene BCR ABL is expressed. The tyrosine kinase activity of BCR-ABL is substantially augmented compared to the tyrosine kinase activity of ABL, and drives the disorders CML, AML and ALL.

The BCR-ABL fusion protein found in Philadelphia chromosome CML patients is a protein of approximately 210 kilodaltons (p210wt or p210BCR-ABL). The fusion transcript typically results from bcr exon 13 or 14 joined to abl exon 2. (Chissoe et al (1995) Genomics 27:67-82). The ABL portion of the p210wt therefore consists of exons 2-11 of the c-abl gene. The common convention in the literature to identify positions in the ABL tyrosine kinase portion in a particular BCR-ABL allele is to use the amino acid numbering for ABL which results from alternative splicing using exon 1a, See GenBank protein ID AAB60394.1. Therefore the mutations referenced herein are likewise keyed to the amino acid numbering of this splice variant whose sequence is provided as ID AAB60394.1 and SEQ ID NO:1.

The first 26 amino acids of SEQ ID NO: 1 are encoded by abl exon 1a and are not found in the BCR-ABL fusion protein p210wt. The amino acid sequence encoded by exon 2 starts with E27 of proto-oncogene abl which results from the transcript which results form the alternative splicing that uses exon 1a. The protein kinase domain is from approximately Ile 242 to amino acid 493. In this numbering system, the phosphate-binding loop (p-loop) is amino acids 249-256, the catalytic region is amino acids 361-367 and the activation loop is amino acids 380-402.

Protein kinases such as ABL and BCR-ABL regulate cell proliferation. Inhibition of protein kinases has been accomplished by administration of ATP-competitive small molecules that block kinase enzymatic activity and thereby interfere with phosphorylation of cellular substrates. Examples of ATP-competitive small molecule inhibitors of kinases include, for example, PD180970, BMS-354825, imatinib, SU5416, SU6668, SU11248, AP23464, gefitinib, erlotinib, PD153035, and SB203580, the structures of which are shown in WO 2006/074149, Table 1.

The ATP-competitive tyrosine kinase inhibitor imatinib is highly effective in the treatment of hematologic neoplasms, particularly CML, AML and ALL. However, ATP-competitive kinase inhibitors such as imatinib have been shown to create selective pressures in target proliferating cells associated with the disorders for which the inhibitors are therapeutically administered. These selective pressures often result in the development of resistance in the target cells. Resistance may arise from mutation of the targeted kinase. Thus, a portion of imatinib-treated patients treated relapse when imatinib-resistant cells emerge.

Imatinib resistance may result from kinase mutations at the site of imatinib binding. Mutations may occur in the kinase catalytic domain, which includes the so-called ‘gatekeeper’ residue, Thr315, and other residues that contact imatinib during binding (e.g., Phe317 and Phe 359). Other mutations occur in the ATP binding domain (the p-loop) and in the activation loop, an area of the kinase structure involved in a conformational change that occurs upon imatinib binding.

Imatinib binding to BCR-ABL involves formation of a hydrogen bond to the Thr315 hydroxyl group. The substitution of isoleucine for threonine at position 315 is one of the most frequent BCR-ABL mutations in imatinib-resistant CML. Alteration of Thr315 to the larger and non-hydrogen bonding isoleucine directly interferes with imatinib binding. Thr315, though necessary for binding of imatinib, is not required for ATP binding to BCR-ABL. Thus, the catalytic activity, and therefore the tumor-promoting function of the BCR-ABL oncoprotein, is preserved in the T315I mutant.

Imatinib binding is also affected by mutations in the kinase p-loop. BCR-ABL sequence analysis in relapsed CML and Ph+ ALL patients has detected p-loop mutations at Tyr253 and Glu255. Amino-acid substitutions at these positions may interfere with the distorted p-loop conformation required for imatinib binding. This is consistent with the observed in vivo resistance and the particularly poor prognosis of patients affected by BCR-ABL p-loop mutations such as Y253F and E255K (Branford et al., Blood, 102, 276-283 (2003).

Imatinib binding is associated with the inactive, unphosphorylated state of the BCR-ABL activation loop. Mutations in this region, such as H396R, destabilize a closed conformation of the activation loop and thereby counteract imatinib inhibition.

Another group of point mutations, remote from the imatinib binding site, lie in the carboxy-terminal lobe of the kinase domain. The most frequently detected BCR ABL mutation falling into this group is M351T. The M351T mutation accounts for 15-20% of all cases of imatinib clinical resistance (Hochhaus et al., Leukemia, 18, 1321-31 (2004). M351T mutation appears to affect the precise positioning of residues in direct contact with imatinib (Cowan-Jacob et al., Mini Rev. Med. Chem., 4, 285-299 (2004), and Shah et al., Cancer Cell, 2, 117-125 (2002).

The above BCR-ABL mutations account for most of the BCR-ABL mutations which have been associated with imatinib resistance. However, a saturating mutational analysis of full-length BCR-ABL combined with a cellular screening procedure selecting for BCR-ABL-driven cell proliferation in the presence of imatinib, has revealed that mutations outside of the kinase domain can weaken kinase interaction with imatinib and thereby contribute to target resistance (Azam, et al., Cell, 112, 831-843 (2003).

BCR-ABL mutations clinically relevant to development of resistance to ATP-competitive kinase inhibitors such as imatinib include the following: G250E, F317L, Y253F, H396R, F311L, M351T, T315I, H396P, E255V, Y253H, Q252H, M244V, L387M, E355G, E255K and F359V. See, von Bubnoff et al., Leukemia 17, 829-838 (2003); Cowan-Jacob, et al., Mini Rev. Med. Chem. 4, 285-299 (2004); Hochhaus, et al., Leukemia 18, 1321-1331 (2004); Nardi, et al., Curr. Opin. Hematol. 11, 35-43 (2004); Ross, et al., Br. J. Cancer 90, 12-19; and Daub et al., Nature Reviews Drug Discovery, 3, 1001-10 (2004).

As of 2009, hematological malignancies accounted for 9.6% of all cancers diagnosed in the United States (“Facts & Statistics”, The Leukemia and Lymphoma Society). Needed are additional agents for treatment of hematological neoplasms, particularly hematological neoplasms that have become resistant to treatment with ATP-competitive small molecule inhibitors of kinases.

### SUMMARY OF THE INVENTION

Provided is a method for treating a hematological neoplasm resistant to treatment with an ATP-competitive inhibitor of BCR-ABL, which resistance results from a mutation of one or more amino acid residues of BCR-ABL. The method comprises administering to a subject in need of such treatment an effective amount of at least one compound selected from the group consisting of N⁶-benzyladenosine, N⁶-benzyladenosine-5′-monophosphate, and pharmaceutically acceptable salts thereof.

In certain embodiments, the hematologic neoplasm is chronic myeloid leukemia, acute myelogenous leukemia or acute lymphoblastic lymphoma. In some embodiments, the method further comprises administering to the individual being treated at least one other chemotherapeutic agent effective against hematologic neoplasms. By “other chemotherapeutic agent effective against hematological neoplasms” is meant a chemotherapeutic agent, other than (i) N⁶-benzyladenosine or (ii) N⁶-benzyladenosine-5′-monophosphate, or pharmaceutically acceptable salts thereof, that is effective in hematological neoplasms. Compounds (i) and (ii) and their pharmaceutically acceptable salts are referred to in this context as “primary anti-hematological neoplasm agent”. In some embodiments, the other chemotherapeutic agent is administered serially with the primary anti-hematological neoplasm agent. In some embodiments, the agents are administered simultaneously. In some embodiments, they are administered in the same dosage form.

According to some embodiments of the method for treating a hematological neoplasm resistant to treatment with an ATP-competitive inhibitor of BCR-ABL, the ATP-competitive inhibitor of BCR-ABL to which the neoplasm has become resistant is imatinib.

In some embodiments, the resistance to treatment with an ATP-competitive inhibitor of BCR-ABL results from a mutation in said BCR-ABL which comprises an alteration of at least one amino acid residue within the BCR-ABL p-loop. In some embodiments, the mutation comprises an alteration of amino acids Tyr 253 or Glu255.

In some embodiments, the resistance to treatment with an ATP-competitive inhibitor of BCR-ABL results from a mutation in said BCR-ABL which comprises an alteration of at least one amino acid residue within the BCR-ABL activation loop. In some embodiments, the mutation comprises an alteration of amino acid His396.

In some embodiments, the resistance to treatment with an ATP-competitive inhibitor of BCR-ABL results from a mutation in said BCR-ABL which comprises at least one mutation selected from the group consisting of F317L, H396R, M351T, H396P, Y253H, M244V, E355G, F359V, G250E, Y253F, F311L, T315I, E255V, Q252H, L387M, and E255K.

In other embodiments,
N⁶-benzyladenosine,
N⁶-benzyladenosine-5′-monophosphate,
or
pharmaceutically acceptable salt thereof,
   is provided for use in:
treating a hematological neoplasm resistant to treatment with an ATP-competitive inhibitor of BCR-ABL, optionally with administration of at least one other chemotherapeutic agent effective against hematological neoplasms.

### ABBREVIATIONS

The following abbreviations may be utilized in the text and the figures:

N6BA or N⁶BA: N⁶-benzyladenosine.

N6BAP or N⁶BAP: 1V6-benzyladenosine-5′-monophosphate.

### BRIEF DESCRIPTION OF THE FIGURES

FIG. 1 shows the domain structure of Stat5.

FIG. 1B shows a binding model of N⁶-benzyladenosine-5′-monophosphate (N6BAP), to the Stat5a SH2-domain.

FIG. 1C shows a binding model of N⁶-benzyladenosine-5′-monophosphate to the Stat5a SH2-domain by stick model where showing the SH2 dimer interface site of Stat5a. Sub-pockets P1, P2, P3 and P1′ of the SH2 interface are labeled.

FIG. 2A shows Western blots of lysates of K562 cells that were treated with N6BAP, the Stat5 inhibitor pimozide, or control. Western blotting of the lysates was carried out using the following as primary antibodies: anti-phosphotyrosine-Stat5a/b (“anti-pYStat5”) mAb or anti-Stat5ab mAb, to show the level of STAT5a/b phosphorylation in the treated cells. Anti-actin was included as a control. FIG. 2A demonstrates that N6BAP blocks phosphorylation of Stat5 in the human BCR-ABL-driven leukemia cell line K562.

FIG. 2B is similar to FIG. 2A, but using anti-phosphotyrosine mAb as the primary antibody. The results show that N6BAP did not affect phosphorylation of BCR-ABL or BCR-ABL protein levels in the K562 cells.

FIG. 3 includes plots of the viability of K562 cells treated with N6BAP or control compound (C5) at the indicated concentrations, assessed by MTS (3-(4,5-dimethylthiazolyl-2)-2,5-diphenyl-tetrazolium bromide) metabolic activity assay. N6BAP reduced the number of viable K563 cells.

FIGS. 4A and 4B are proliferation assay plots of the K562 parent cell line (4A) and an imatinib-resistant K562 line (4B) cultured in the presence of culture medium without any compounds (-♦-, Ctrl), DMSO (-▪-, 0.1%), the control compound C5 (-▴-, 5 μM) or N6BAP (-X-, 5 μM). Cells were harvested at 24, 48, 72 and 96 hours, trypan blue stained and the numbers of viable cells were counted for each time-point. N6BAP blocked the growth of the parental K562 line (4A) and the imatinib-resistant cell line (4B).

FIGS. 5A-5D are plots of the culturing of parent cell lines KCL22 (FIG. 5A) and BV173 (FIG. 5C), and the corresponding imatinib-resistant cell lines containing the T315I BCR-ABL mutations, KCL22 T315I (FIG. 5B) and BV173 T315I (FIG. 5D), in the presence of: of culture medium without any compounds (-♦-, Ctrl), DMSO (-▪-, 0.1%), the control compound C5 (-▴-, 5 μM) or N6BAP (-X-, 5 μM) culture medium without any compounds (Ctrl), DMSO (0.1%), the control compound C5 (5 μM) or N6BAP (5 μM). Cells were harvested at 24, 48, 72 and 96 hours, trypan blue stained and the numbers of viable cells were counted for each time-point. N6BAP blocked the growth of not only the parental cell lines (FIGS. 5A, 5C) but also the growth of the corresponding imatinib-resistant cell lines (FIGS. 5B, 5D).

FIG. 5E shows Western blots of parental KCL22 cells (KCL22S) and imatinib-resistant (KCL22R) cells treated with N6BAP at various doses. Western blotting of the lysates was carried out using the following as primary antibodies: anti-phosphotyrosine-Stat5a/b (“anti-pYStat5”) mAb or anti-Stat5ab mAb, to show the level of STATSa/b phosphorylation in the treated cells. Anti-actin was included as a control. FIG. 5E demonstrates that N6BAP blocks phosphorylation of Stat5 in the human BCR-ABL-driven parental KCL22 cells and in imatinib-resistant KCL22 cells.

FIG. 6 shows the effect of varying concentration of the compound N⁶-benzylodenosine (“N6BA”) in the transcriptional activity of Stat5 on the β-casein gene promoter in human prostate PC-3 cells in a luciferase reporter gene assay. The cells were transfected with pStat5a or pStat5 b, pPrlR (prolactin receptor) plasmids, 0.5 μg of pBeta-casein-luc and 0.025 μg of pRL-TK (Renilla luciferase) plasmids as an internal control. The results, shown in FIG. 6, demonstrate that N6BA inhibits transcriptional activity of Stat5.

FIG. 7A comprises Western blots of lysates of K562 cells that were treated with N6BA or control compound at the indicated concentrations for 3 or 6 hours. Western blotting of the lysates was carried out using antibodies to phosphotyrosine-Stat5a/b (lanes marked “pStat5”) and Stat5ab (lanes marked “Stat5”). Anti-actin was included as a control. N6BA inhibited phosphorylation of Stat5 in the K562 cells.

FIG. 7B comprises Western blots of lysates of imatinib-sensitive (KCL22S) or imatinib-sensitive (KCL22R) cells that were treated with compound N6BA or control compound at the indicated concentrations for 6 or 16 hours. Western blotting of the lysates was carried out using antibodies to phosphotyrosine-Stat5a/b (lanes marked “pStat5”) and Stat5ab (lanes marked “Stat5”). Anti-actin was included as a control. N6BA inhibited phosphorylation of Stat5 in both the imatinib-sensitive and imatinib-resistant KCL22 cells.

FIG. 8 shows the result of a study demonstrating that N6BA blocks dimerization of Stat5a/b. DMSO and the control compound C5 were included in the study.

FIGS. 9A and 9B are plots showing that compound N6BA reduces the number of K562 leukemia cells (9A) vs. the control compound, C5, (9B).

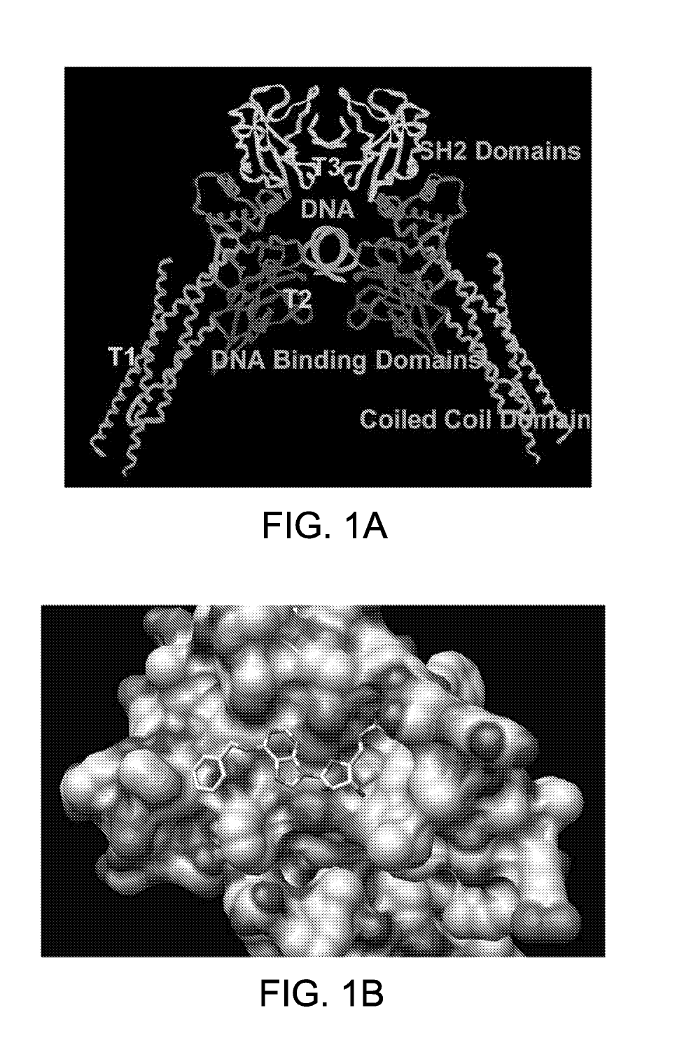

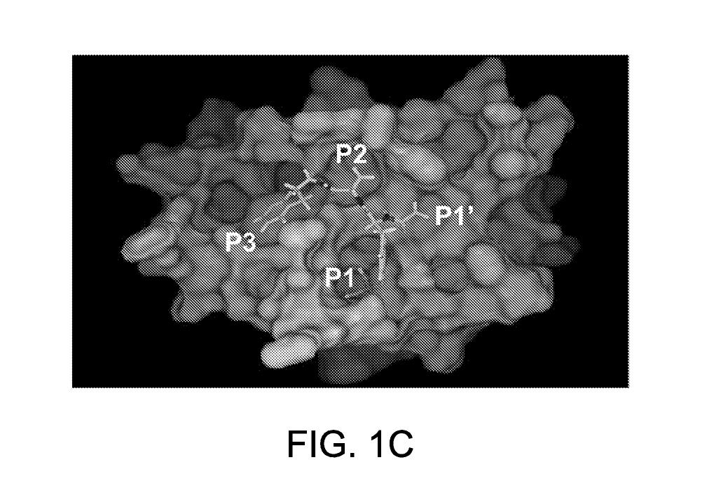

### DETAILED DESCRIPTION OF THE INVENTION

As used in the specification and the appended claims, the singular forms “a,” “an” and “the” include plural referents unless the context clearly dictates otherwise.

As used herein, the terms “treat” and “treatment” are used interchangeably and are meant to indicate a postponement of development of a disorder and/or a reduction in the severity of symptoms that will or are expected to develop. The terms further include ameliorating existing symptoms, preventing additional symptoms, and ameliorating or preventing the underlying metabolic causes of symptoms.

The expression “effective amount” or “therapeutically effective amount” when used to describe therapy to an individual suffering from a hematological neoplasm, refers to the amount of a compound that inhibits the growth or proliferation of cells of a hematological neoplasm, or alternatively induces apoptosis of such cells, preferably resulting in a therapeutically useful and preferably selective cytotoxic effect on the cells.

As used herein, the term “subject” or “patient” refers to any animal (e.g., a mammal), including, but not limited to humans, non-human primates, rodents, and the like. Typically, the terms “subject” and “patient” are used interchangeably herein in reference to a human subject.

The expression “kinase inhibitor” refers to an agent that acts to inhibit the kinase activity of a kinase.

The expression “ATP-competitive kinase inhibitor” means a kinase inhibitor that inhibits the kinase by competing with ATP for the ATP binding site on the kinase.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

N⁶-benzyladenosine has the following chemical structure:

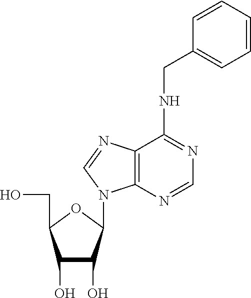

N⁶-benzyladenosine-5′-monophosphate has the following chemical structure:

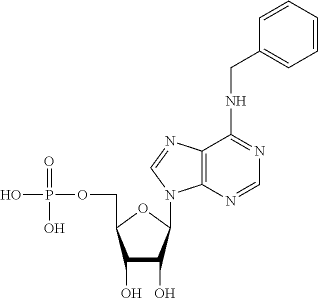

N⁶-benzyladenosine and its 5′-monophosphate may be prepared as described in the literature.

According to the present invention, it has been found that N⁶-benzyladenosine and N⁶-benzyladenosine 5′-monophosphate inhibit Stat5a/b phosphorylation of neoplastic cells of hematological neoplasms driven by expression of the oncogene BCR-ABL, such as the hematological neoplasm CML, inhibits the viability and proliferation of such cells, and induces their apoptotic death. It has further been found that such effects are apparent even in hematological neoplasm that display resistance to treatment with an ATP-competitive inhibitor of BCR-ABL, wherein the resistance results from a mutation of one or more amino acid residues of BCR-ABL.

In the activation cascade of Stat5a/b, the molecule first becomes phosphorylated at a conserved tyrosine residue in its C-terminus by an upstream tyrosine kinase (such as Jak2 or Src). Phosphorylation of Stat5a/b leads to dimerization of Stat5a/b, and the dimerized Stat5a/b translocates to the nucleus followed by binding of dimerized Stat5a/b to the promoters of its target genes to regulate transcription. Dimerization of Stat5a/b is required for Stat5a/b to bind DNA and exert is transcriptional activity. According to the present invention, the compounds described herein block Stat5a/b dimerization.

Translocation of Stat5a/b to the nucleus is required in order for that molecule to exert its transcriptional activity. In the nucleus, Stat5a/b bind to specific Stat5a/b response elements of target gene promoters. According to the present invention, compounds described herein block Stat5a/b translocation to the nucleus of cancer cells. The compounds described herein inhibit proliferation of cells of hematological neoplasms, and induce apoptosis of those cells, as demonstrated in the assays described below.

The compounds described herein may be used for the treatment of hematologic neoplasms, of all types, that are resistant to treatment with an ATP-competitive inhibitor of BCR-ABL. Without limitation, the compounds described herein are particularly useful in the treatment of the BCR-ABL-driven hematologic neoplasms, including without limitation CML and ALL. By “BCR-ABL-driven” is meant a neoplasm which is characterized by the presence of the BCR-ABL oncogene, as for example, in AML, ALL and CML.

Treatment of hematological neoplasm resistant to treatment with an ATP-competitive inhibitor of BCR-ABL may be carried out by following detection of BCR-ABL mutations in neoplastic cells of the subject. Mutations may be detected, for example, by sequencing the subject's bcr-abl gene in a leukemic cell, and comparing the sequence against a databank of resistance-conferring mutations. In addition to direct DNA sequencing, the following methods known to those skilled in the art may also be used to detect kinase mutations: Single-strand Conformation Polymorphism (SSCP); Denaturing Gradient Gel Electrophoresis (DGGE); Denaturing High-Performance Liquid Chromatography (DHPLC); Chemical Mismatch Cleavage (CMC); Enzyme Mismatch Cleavage (EMC); Heteroduplex analysis; and the use of DNA microarrays.

According to one embodiment of the invention, quantitative polymerase chain reaction (RQ-PCR) of BCR-ABL mRNA in imatinib-treated subjects may be used to detect patients at risk of resistance. A significant portion of imatinib-treated subjects displaying a two-fold or more increase in bcr-abl expression have detectable BCR-ABL mutations, indicating that such a rise in BCR-ABL may serve as a primary indicator to test patients for imatinib-deactivating BCR-ABL kinase domain mutations (Branford et al., Blood, 104, 2926-32 (2004)). Elevated BCR-ABL expression in the cells of a subject undergoing ATP-competitive kinase inhibitor therapy would suggest the need for mutation analysis to identify possible resistance-conferring BCR-ABL mutations, particularly in the BCR-ABL kinase domain.

According to some embodiments of the method for treating a hematological neoplasm resistant to treatment with an ATP-competitive inhibitor of BCR-ABL, the ATP-competitive inhibitor of BCR-ABL to which the neoplasm has become resistant is imatinib.

The resistant hematological neoplasm treated may result from a mutation in BCR-ABL which comprises an alteration of at least one amino acid residue within the BCR-ABL p-loop, e.g., amino acids Tyr 253 or Glu255. Alternatively, resistance may arise from an alteration of at least one amino acid residue within the BCR-ABL activation loop, e.g., an alteration of amino acid His396. In other embodiments, resistance may result from a mutation in BCR-ABL which comprises at least one mutation selected from the group consisting of F317L, H396R, M351T, H396P, Y253H, M244V, E355G, F359V, G250E, Y253F, F311L, T315I, E255V, Q252H, L387M, and E255K.

N⁶-benzyladenosine and N⁶-benzyladenosine-5′-monophosphate may be converted to a salt for use according to the present invention. The term “pharmaceutically acceptable salt” refers to salts which possess toxicity profiles within a range that affords utility in pharmaceutical applications.

Suitable pharmaceutically-acceptable salts may take the form of base addition salts that may include, for example, metallic salts, e.g., alkali metal, alkaline earth metal and transition metal salts such as, for example, calcium, magnesium, potassium, sodium and zinc salts. The ammonium salt is a preferred salt.

Pharmaceutically acceptable base addition salts also include organic salts made from basic amines such as, for example, N,N-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. These salts may be prepared by conventional means from the subject compounds by reaction with the appropriate base.

The compounds used in the methods of the present invention may be administered by any route, including oral and parenteral administration. Parenteral administration includes, for example, intravenous, intramuscular, intraarterial, intraperitoneal, intranasal, rectal, intravesical, intradermal, topical or subcutaneous administration. Also contemplated within the scope of the invention is the instillation of drug in the body of the patient in a controlled formulation, with systemic or local release of the drug to occur at a later time. For example, the drug may be localized in a depot for controlled release to the circulation, or for release to a local site of tumor growth.

The specific dose of compound to obtain therapeutic benefit for treatment of a proliferative disorder will, of course, be determined by the particular circumstances of the individual patient including, the size, weight, age and sex of the patient, the stage of the disease, the aggressiveness of the disease, and the route of administration of the compound.

For example, a daily dosage of from about 0.01 to about 50 mg/kg/day may be utilized, or from about 1 to about 40 mg/kg/day, or from about 3 to about 30 mg/kg/day. Higher or lower doses are also contemplated. A therapeutically effective amount may also be estimated on the basis of the in vitro and in vivo studies hereinafter described.

The daily dose of the compound may be given in a single dose, or may be divided, for example into two, three, or four doses, equal or unequal, but preferably equal, that comprise the daily dose. When given intravenously, such doses may be given as a bolus dose injected over, for example, about 1 to about 4 hours.

The compounds used in the methods of the present invention may be administered in the form of a pharmaceutical composition, in combination with a pharmaceutically acceptable carrier. The active ingredient in such formulations may comprise from 0.1 to 99.99 weight percent. By “pharmaceutically acceptable carrier” is meant any carrier, diluent or excipient which is compatible with the other ingredients of the formulation and not deleterious to the recipient.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The pharmaceutical composition is preferably in unit dosage form. In such form the preparation is divided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds used in the methods of the present invention may be administered according to the invention in combination with one or more other active agents, for example, a least one other anti-proliferative compound, or drug to control side-effects, for example anti-emetic agents. The further active agent may comprise, for example a chemotherapeutic agent effective against hematological neoplasms. Such other agents include, for example, targeted therapies such as imatinib, dasatinib, nilotinib or nelarabine. Further, such chemotherapeutic agents for the treatment of hematological neoplasms include vincristine, daunorubicine, idarubicine, cytarabine, L-asparaginase, etoposide, teniposide, 6-mercaptopurine, methotrexate, cyclophosphamide, prednisone, 6-thioguanine, hydroxyurea, ATRA, ATO, fludarabine, pentostatin, cladribine, chlorambucil, bendamustine, oxaliplatin, etoposide, topotecan, azacytidine, and decitabine. Monoclonal antibodies for the treatment of hematological neoplasms include rituximab, alemtuzumab and ofatumumab.

In one embodiment of the invention, the combination of N⁶-benzyladenosine or N⁶-benzyladenosine-5′-monophosphate, or pharmaceutically acceptable salt thereof, and the other anticancer agent, are co-formulated and used as part of a single pharmaceutical composition or dosage form. The compositions according to this embodiment of the invention comprise one or more of the aforementioned primary agent, and at least one other chemotherapeutic agent, in combination with a pharmaceutically acceptable carrier. In such compositions, the primary agent, and the second chemotherapeutic agent, may together comprise from 0.1 to 99.99 weight percent of the total composition. The compositions may be administered by any route and according to any schedule which is sufficient to bring about the desired antiproliferative effect in the patient.

According to other embodiments of the invention, the combination of the primary anticancer agent N⁶-benzyladenosine or N⁶-benzyladenosine-5′-monophosphate or pharmaceutically acceptable salt thereof, and the at least one other chemotherapeutic agent, may be formulated and administered as two or more separate compositions, at least one of which comprises the primary anti cancer agent, and the other comprises the other chemotherapeutic agent. The separate compositions may be administered by the same or different routes, administered at the same time or different times, and administered according to the same schedule or on different schedules, provided the dosing regimen is sufficient to bring about the desired antiproliferative effect in the patient. When the drugs are administered in serial fashion, it may prove practical to intercalate administration of the two drugs, wherein a time interval, for example a 0.1 to 48 hour period, separates administration of the two drugs.

The practice of the invention is illustrated by the following non-limiting examples.

### EXAMPLES

Cells used in the following procedures were cultured according to the following conditions. Leukemia cells (K562, K562R, KCL22, KCL22R, BV173 and BV173R) were cultured in RPMI 1640 containing penicillin/streptomycin and 10% fetal bovine serum (FBS. Cells were maintained in a 37° C. humidified incubator with a mixture of 95% air and 5% CO₂.

The following is the structure of the compound designated as “C5”, used as a control in certain of the following examples:

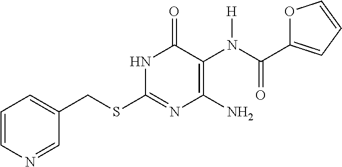 [SMILES: Nc1nc(SCc2cccnc2)[nH]c(=O)c1NC(=O)c1ccco1]

### Example 1

### N⁶-Benzyladenosine-5′-Monophosphate Effect on Phosphorylation of Stat5 and BCR-ABL in K562 Cells

The following experiment demonstrates that N6BAP inhibits constitutive Stat5a/b phosphorylation, but does not affect BCR-ABL phosphorylation, in human chronic myeloid leukemia (K562) cells driven by BCR-ABL. Exponentially growing K562 cells were treated with 5 μM N6BAP or pimozide for 3 h after which the cells were harvested. Pimozide is a Stat5 inhibitor in leukemia cells (Nelson et al., Blood 117:3421-3429, 2011). Cell pellets were solubilized in lysis buffer [10 mM Tris-HCl (pH 7.6), 5 mM EDTA, 50 mM sodium chloride, 30 mM sodium pyrophosphate, 50 mM sodium fluoride, 1 mM sodium orthovanadate, 1% Triton X-100, 1 mM phenylmethylsulfonyl fluoride, 5 μg/ml aprotinin, 1 μg/ml pepstatin A, and 2 μg/ml leupeptin], rotated end-over-end at 4° C. for 60 min, and insoluble material was pelleted at 12,000×g for 30 min at 4° C. The protein concentrations of clarified cell lysates were determined by simplified Bradford method (Bio-Rad Laboratories, Hercules, Calif.). Western blotting of the lysates was carried out using, as primary antibodies, the following antibodies at the following concentrations: anti-phosphotyrosine-Stat5a/b (Y694/Y699) mAb (1 μg/ml, Advantex BioReagents, Conroe, Tex.), anti-Stat5ab mAb (1:250; BD Biosciences, San Jose, Calif.), anti-human Abl mAb (Calbiochem) or anti-phosphotyrosine mAb (Millipore).

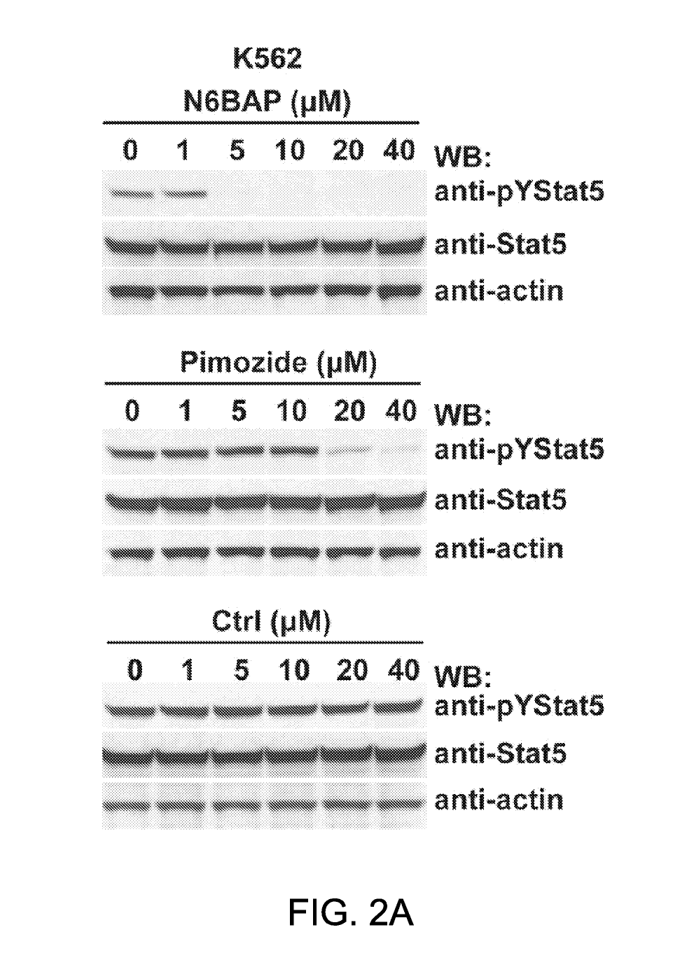

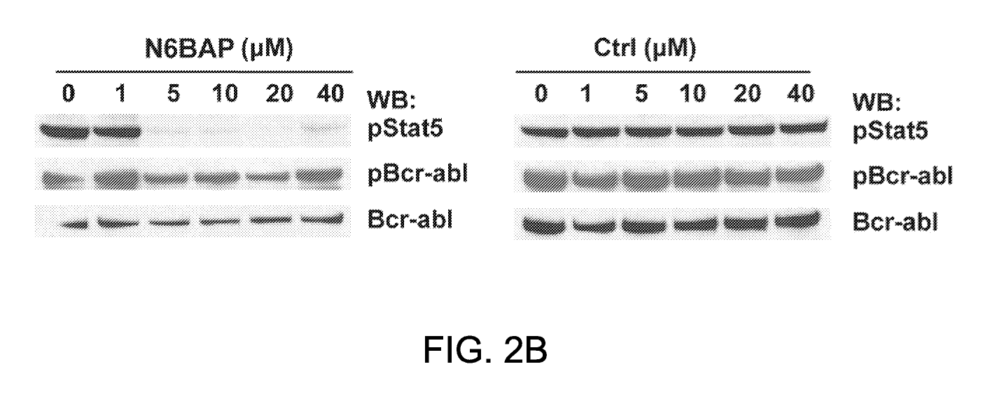

The results are shown in FIGS. 2A and 2B. FIG. 2A demonstrates that N6BAP blocks phosphorylation of Stat5 in the human BCR-ABL-driven leukemia cells. FIG. 2B show that N6BAP did not affect phosphorylation of BCR-ABL or BCR-ABL protein levels in the same human leukemia cells.

### Example 2

### N⁶-Benzyladenosine-5′-Monophosphate Effect on K562 Cell Viability

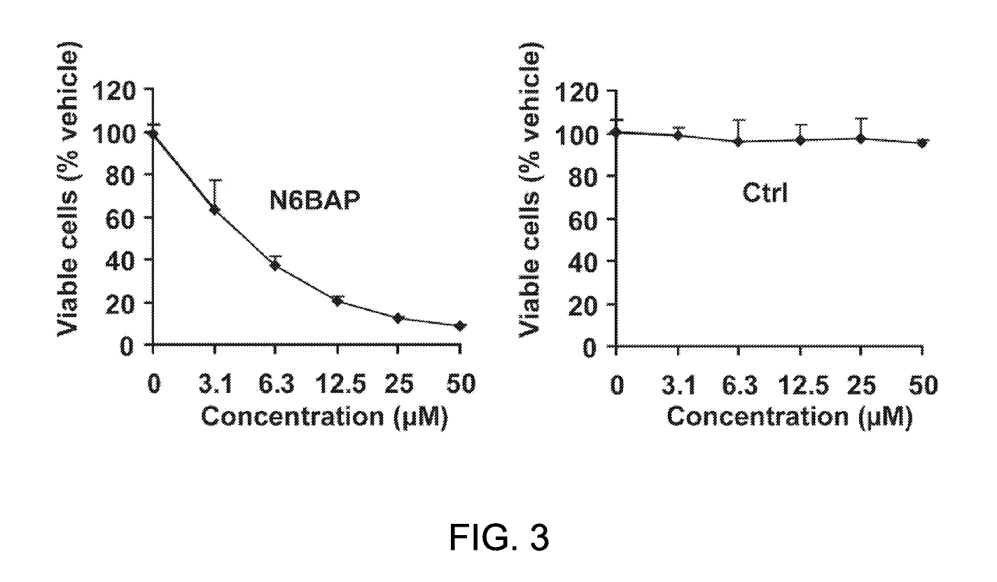

Exponentially growing K562 cells were treated with N6BAP or the control compound C5 for 72 h at 3.1, 6.3, 12.5, 25 and 50 μM indicated concentrations. The cell viability was assessed by MTS (3-(4,5-dimethylthiazolyl-2)-2,5-diphenyl-tetrazolium bromide) metabolic activity assay (CellTiter 96® AQueous Assay kit). The results, shown in FIG. 3, demonstrate that N6BAP reduces the number of viable K562 human leukemia cells.

### Example 3

### N⁶-Benzyladenosine-5′-Monophosphate Growth Inhibition of Imatinib-resistant K562 Cell Viability

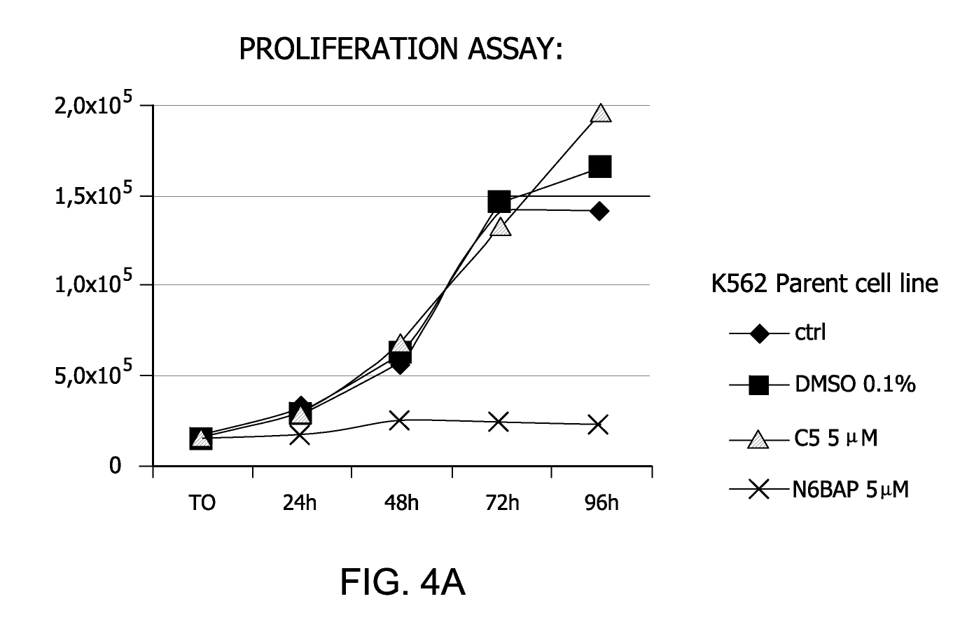

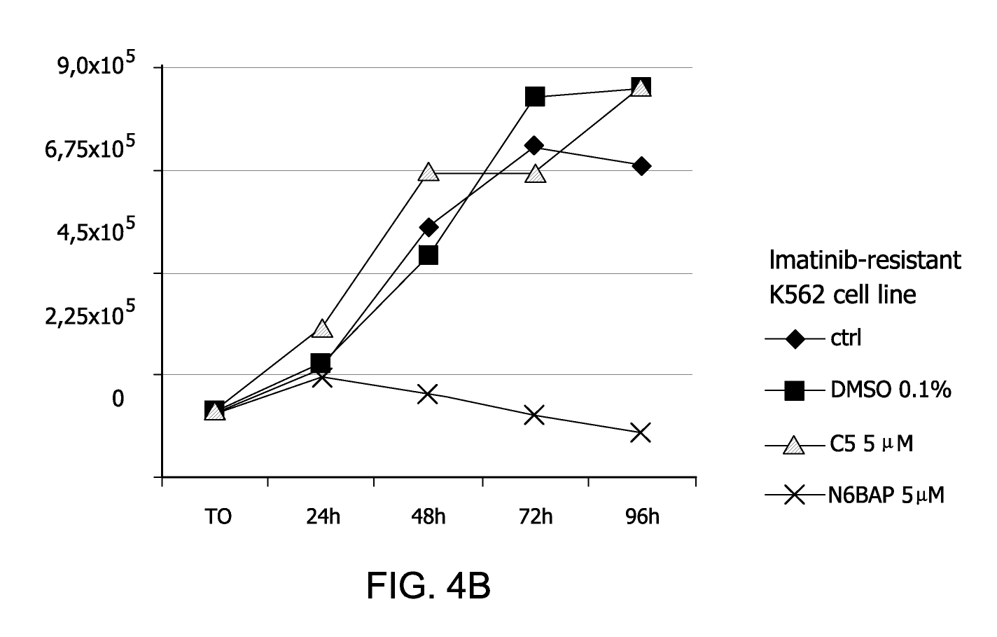

The K562 parent cell line and an imatinib-resistant K562 line were cultured in the presence of culture medium without any compounds (Ctrl), DMSO (0.1%), the control compound C5 (5 μM) or N6BAP at 5 μM concentration. The cells were harvested at 24, 48, 72 and 96 hours, trypan blue stained and the numbers of viable cells were counted for each time-point. The results are shown in FIGS. 4A and 4B. N6BAP blocked not only the growth of the parental K562 line (FIG. 4A), but also blocked the growth of the imatinib-resistant cell line (FIG. 4B).

### Example 4

### N⁶-Benzyladenosine-5′-Monophosphate Growth Inhibition of Other Imatinib-resistant BCR-ABL-Driven Human Leukemic Cell Lines

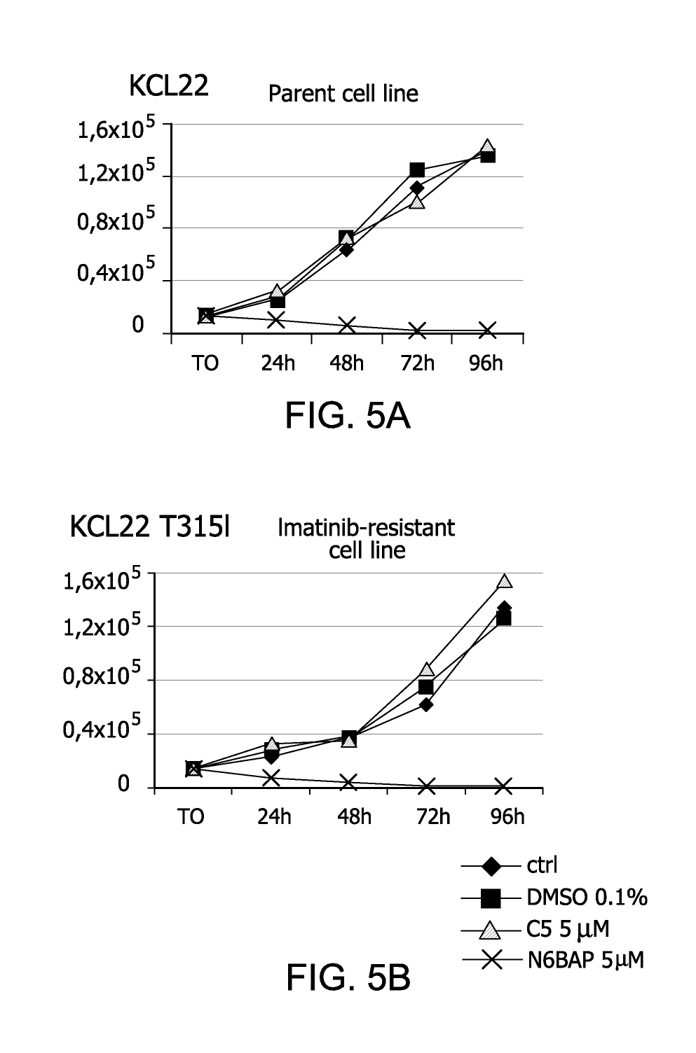

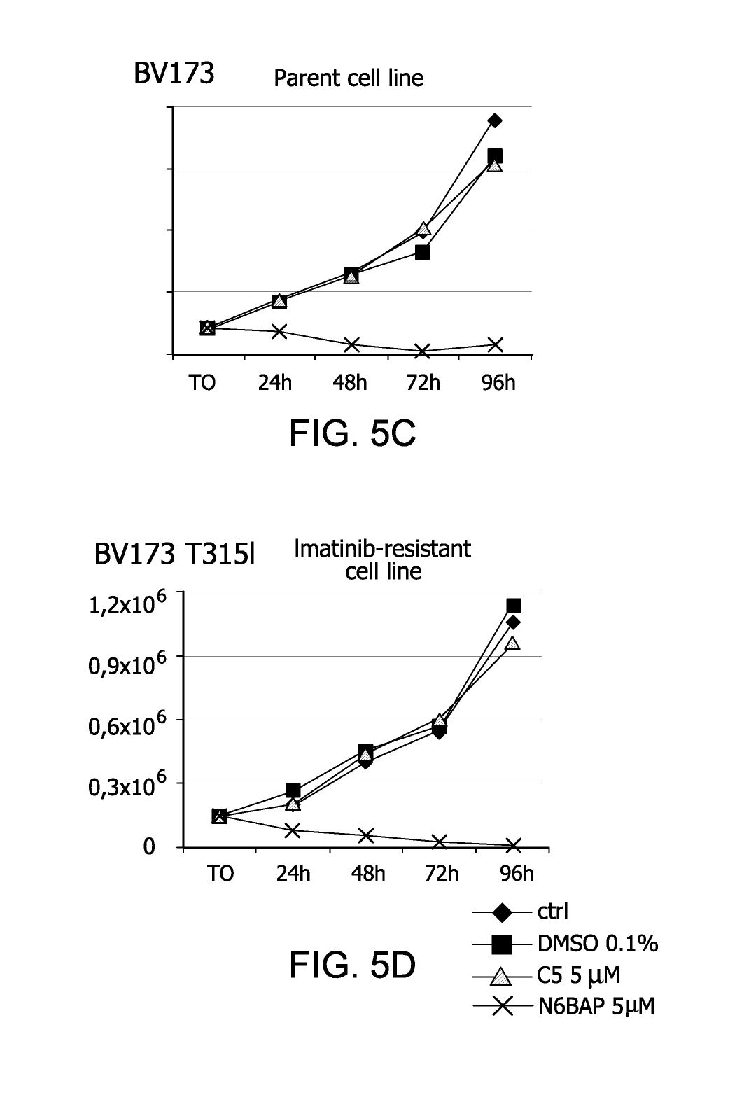

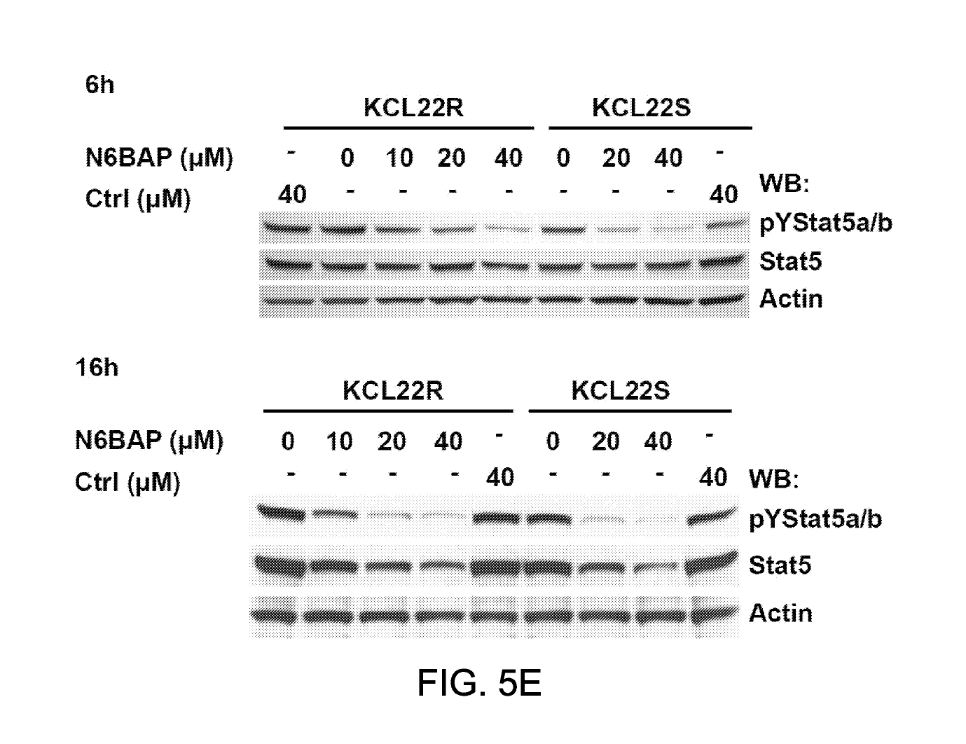

KKCL22 and BV173 parent cell lines, and imatinib-resistant counterpart cell lines, were cultured in the presence culture medium without any compounds (Ctrl), DMSO (0.1%), the control compound C5 (5 μM) or N6BAP at 5 μM concentration. The imatinib-resistant lines are characterizes by the bcr-abl T315I mutation, which provides imatinib resistance. The cells were harvested at 24, 48, 72 and 96 hours, trypan blue stained and the numbers of viable cells were counted for each time-point. The results are shown in FIGS. 5A-D. N6BAP blocked not only the growth of the parental cell lines KCL22 (FIG. 5A) and BV173 (FIG. 5C), but also blocked the growth of the corresponding imatinib-resistant cell lines containing the T315I BVR-ABL mutations, KCL22 T315I (FIG. 5B) and BV173 T315I (FIG. 5D). A cell cycle progression analysis (data not shown) indicated that N6BAP induced apoptotic death.

### Example 5

The following experiment demonstrates that N6BAP inhibits constitutive Stat5a/b phosphorylation in human chronic myeloid leukemia (KCL22) and imatinib resistant KCL22R cells driven by BCR-ABL. Exponentially growing KCL22 and KCL22R cells were treated with N6BAP at various concentrations for 6 h or 16 h after which the cells were harvested. Cell pellets were solubilized in lysis buffer [10 mM Tris-HCl (pH 7.6), 5 mM EDTA, 50 mM sodium chloride, 30 mM sodium pyrophosphate, 50 mM sodium fluoride, 1 mM sodium orthovanadate, 1% Triton X-100, 1 mM phenylmethylsulfonyl fluoride, 5 μg/ml aprotinin, 1 μg/ml pepstatin A, and 2 μg/ml leupeptin], rotated end-over-end at 4° C. for 60 min, and insoluble material was pelleted at 12,000×g for 30 min at 4° C. The protein concentrations of clarified cell lysates were determined by simplified Bradford method (Bio-Rad Laboratories, Hercules, Calif.). Western blotting of the lysates was carried out using, as primary antibodies, the following antibodies at the following concentrations: anti-phosphotyrosine-Stat5a/b (Y694/Y699) mAb (1 μg/ml, Advantex BioReagents, Conroe, Tex.), anti-Stat5ab mAb (1:250; BD Biosciences, San Jose, Calif.) or anti-actin pAb (Sigma). Anti-actin was included as a control.

The results, shown in FIG. 5E, demonstrates that N6BAP blocks phosphorylation of Stat5 in the human BCR-ABL-driven parental KCL22 cells (KCL22S) and in imatinib-resistant KCL22 cells (KCL22R).

### Example 6

### N⁶-benzyladenosine Inhibition of Stat5 Transcriptional Activity

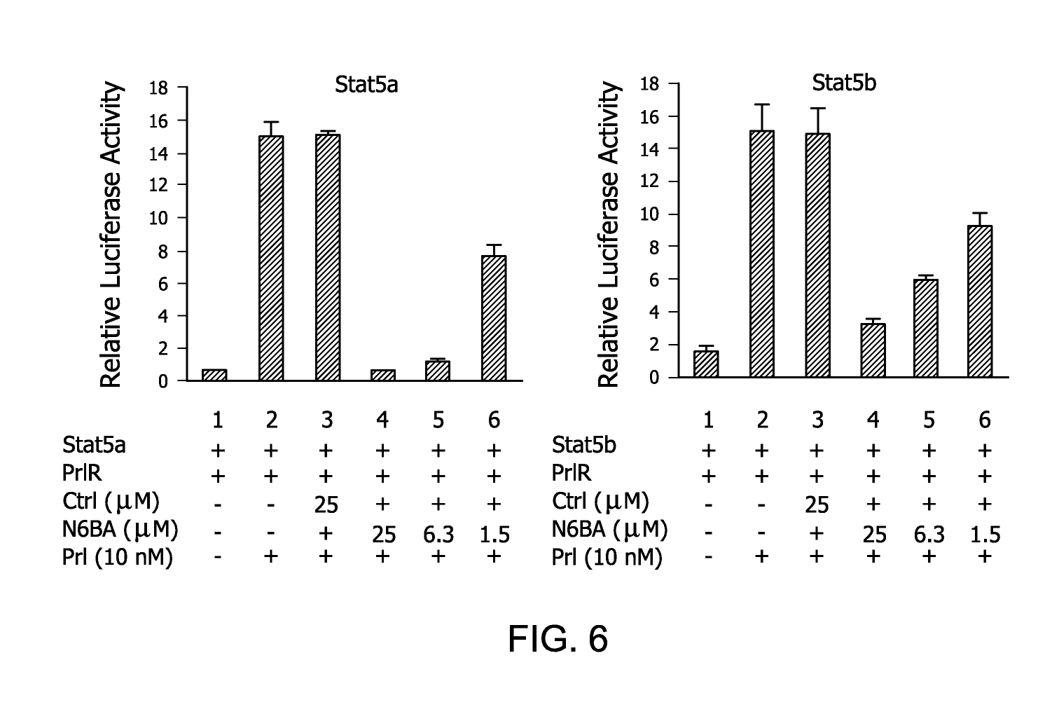

Cells of the human prostate cell line PC-3 were plated into 96-well plate at the density of 2×10⁵ per well. After 24 hours of plating, cells were transiently co-transfected using FuGENE6 (Roche) with 0.25 μg of each of pStat5a or pStat5b, pPr1R (prolactin receptor) plasmids, 0.5 μg of pBeta-casein-luc and 0.025 μg of pRL-TK (Renilla luciferase plasmids as an internal control. After another 24 hours of transfection, the cells were starved in serum-free medium for 8 hours, pretreated with N⁶-benzyladenosine (“N6BA”), or the control compound C5, for 1 hour, and then stimulated with 10 nM human prolactin (hPrl) in the serum-free medium for additional 16 hours. The lysates were assayed for firefly and Renilla luciferase activities using the Dual-Luciferase reporter assay system (Promega). Three independent experiments were carried out in triplicate. The firefly luciferase activity was normalized to the Renilla luciferase activity of the same sample, and the mean was calculated from the parallels. From the mean values of each independent run, the overall mean and its standard deviation (S.D.) were calculated. The results, shown in FIG. 6, demonstrate that N6BA inhibits transcriptional activity of Stat5.

### Example 7

### N⁶-benzyladenosine Inhibits Constitutive Stat5a/b Phosphorylation in K562 Cells

Exponentially growing K562 cells were treated with N6BA for 3 h or 6 h after which the cells were harvested. Exponentially growing KCL22 cells or imatinib-resistant KCL22 cells were treated with NBPP for 6 h or 16 h after which the cells were harvested. Cell pellets were solubilized in lysis buffer [10 mM Tris-HCl (pH 7.6), 5 mM EDTA, 50 mM sodium chloride, 30 mM sodium pyrophosphate, 50 mM sodium fluoride, 1 mM sodium orthovanadate, 1% Triton X-100, 1 mM phenylmethylsulfonyl fluoride, 5 μg/ml aprotinin, 1 μg/ml pepstatin A, and 2 μg/ml leupeptin], rotated end-over-end at 4° C. for 60 min, and insoluble material was pelleted at 12,000×g for 30 min at 4° C. The protein concentrations of clarified cell lysates were determined by simplified Bradford method (Bio-Rad Laboratories, Hercules, Calif.). Western blotting of the lysates was carried out, using as primary antibodies the following antibodies at the following concentrations: anti-phosphotyrosine-Stat5a/b (Y694/Y699) mAb (1 μg/ml, Advantex BioReagents, Conroe, Tex.), anti-Stat5ab mAb (1:250; BD Biosciences, San Jose, Calif.), anti-human Abl mAb (Calbiochem) or anti-phosphotyrosine mAb (Millipore).

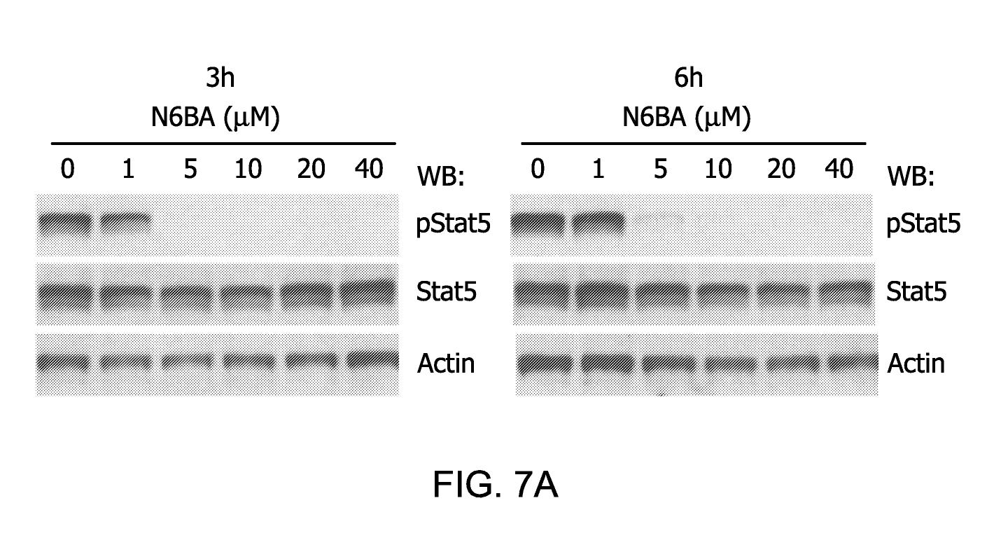

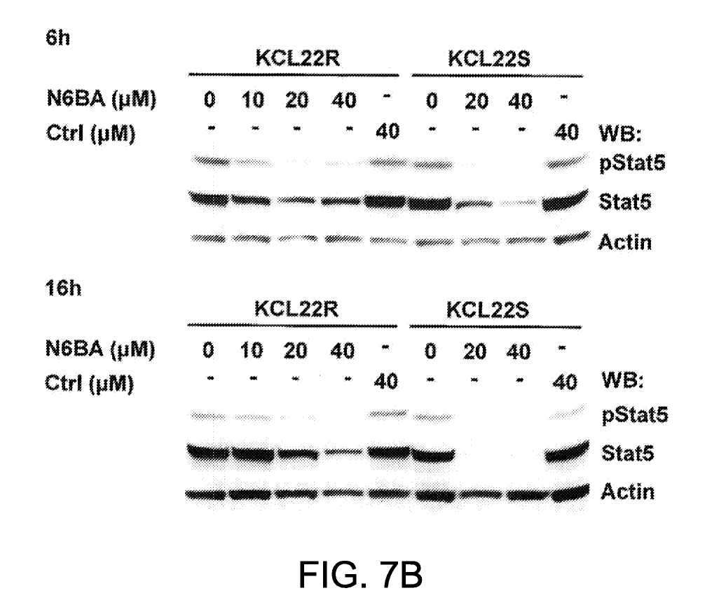

The results for K562 cells are shown in FIG. 7A. The results for KCL22 cells (KCL22S) and imatinib-resistant KCL22 cells (KCL22R) are shown in FIG. 7B. The results demonstrate that N6BA inhibits phosphorylation of Stat5 in imatinib-sensitive leukemic cells, and also in imatinib-resistant leukemic cells.

### Example 8

### N⁶-benzyladenosine Blockage of Stat5 Dimerization

FLAG-tagged Stat5a and MYC-tagged Stat5a were generated as follows by standard molecular biology cloning techniques. Plasmid pCMV3-FLAG-Stat5a, pCMV3-MYC-Stat5a and pPr1R were co-transfected using FuGENE6 (Roche) into PC-3 cells (2 μg of each plasmid per 1×10⁷ cells). The cells were starved for 20 hours, pre-treated with N⁶-benzyladenosine for 2 h, then stimulated with hPrl (10 nM) in RPMI 1640 without serum for 30 minutes. The cell lysates were immunoprecipitated with 25 μl anti-FLAG M2 polyclonal affinity gel (2 μg/ml, Sigma), anti-MYC pAb (1 μg/ml, Upstate) or normal rabbit serum (NRS). The primary antibodies were used in the immunoblottings at the following concentrations: anti-FLAG pAb (1:1000; Stratagene), anti-MYC mAb (1:1000; Sigma), anti-Stat5a/b mAb (1:250) (Transduction Laboratories) detected by horseradish peroxidase-conjugated secondary antibodies.

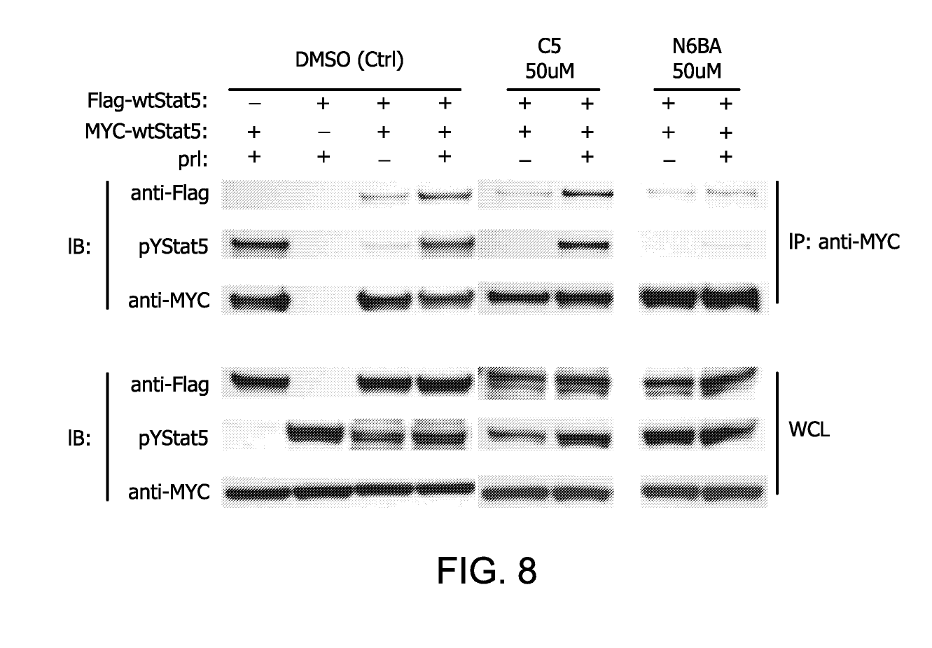

The results are shown in FIG. 8. Lane 1 of FIG. 8 demonstrates cells transfected only with MYC-tagged Stat5a (the third panel from the bottom). Lane 2 demonstrates cells transfected only with FLAG-tagged Stat5a (the second panel from the bottom). Lanes 3-10 demonstrate cells transfected with both FLAG-tagged Stat5a and MYC-tagged-Stat5a (the second and third panels from the bottom, respectively). Lanes 1, 2, 4, 6 and 8 represent cells stimulated with human prolactin (Prl) for 30 minutes which induces dimerization of Stat5. Two hours prior to Prl-stimulation, the cells had been treated with DMSO (lanes 1-4), the control compound C5 (lanes 5 and 6) or N⁶-benzyladenosine (lanes 7 and 8) to test whether the Stat5a/b-inhibitor compound, N6BA, would be able to inhibit dimerization of Stat5. When MYC-tagged Stat5a was immunoprecipitated from the cells (the third panel from the top) and immunoblotted with anti-FLAG pAb (top panel) the control compound did not inhibit dimerization of Stat5a (lane 6). In contrast, N6BA (lane 8) effectively inhibited Stat5a dimerization (lane 8) (=very weak band in Prl-stimulated cells). It should be noted the drug effect in this study was not due to cell apoptosis induction, as the treatment time was only 2 hours.

### Example 9

### N⁶-benzyladenosine Reduces the Viability of K562 Human Leukemic Cells

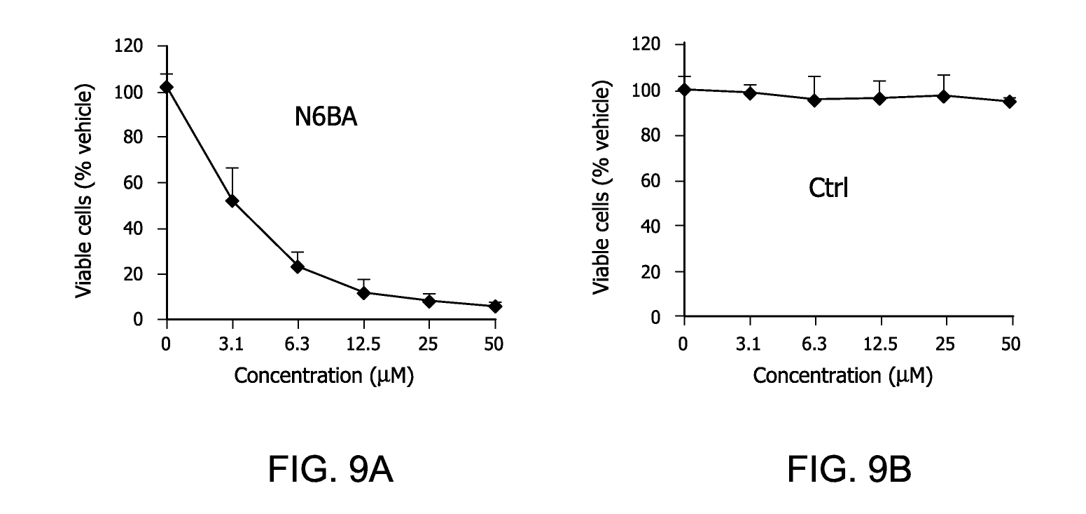

Exponentially growing K562 leukemia cells were treated with N6BA or the control compound C5 for 72 h at 3.1, 6.3, 12.5, 25 and 50 μM concentrations. The cell viability was assessed by MTS (3-(4,5-dimethylthiazolyl-2)-2,5-diphenyl-tetrazolium bromide) metabolic activity assay (CellTiter 96® AQueous Assay kit). The test compound decreased the number of K562 cells (FIG. 9A) compared to the control compound (FIG. 9B).

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

1. A method for treating a hematological neoplasm resistant to treatment with an ATP-competitive inhibitor of BCR-ABL, which resistance results from a mutation of one or more amino acid residues of BCR-ABL, said method comprising administering to a subject in need of such treatment an effective amount of at least one compound selected from the group consisting of:
N⁶-benzyladenosine;
N⁶-benzyladenosine-5′-monophosphate; and
pharmaceutically acceptable salts thereof.

2. The method according to claim 1 wherein the hematologic neoplasm is chronic myeloid leukemia, acute myelogenous leukemia or acute lymphoblastic lymphoma.

3. The method according to claim 2 wherein the ATP-competitive inhibitor of BCR-ABL is imatinib.

4. The method according to claim 1, 2 or 3, wherein the mutation comprises alteration of at least one amino acid residue within the BCR-ABL p-loop.

5. The method according to claim 4 wherein the mutation comprises an alteration of amino acids Tyr 253 or Glu255.

6. The method according to claim 1, 2 or 3, wherein the mutation comprises alteration of at least one amino acid residue within the BCR-ABL activation loop.

7. The method according to claim 6, wherein the mutation comprises an alteration of His396.

8. The method according to claim 1, 2 or 3, wherein the mutation comprises at least one mutation selected from the group consisting of F317L, H396R, M351T, H396P, Y253H, M244V, E355G, F359V, G250E, Y253F, F311L, T315I, E255V, Q252H, L387M, and E255K.

9. The method according to claim 1, 2 or 3, further comprising administering to said individual at least one other chemotherapeutic agent effective against said hematologic neoplasm.

10. The method according to claim 9, wherein said other chemotherapeutic agent is administered serially with said N⁶-benzyladenosine, N⁶-benzyladenosine-5′-monophosphate, or pharmaceutically acceptable salt thereof.

11. The method according to claim 9, wherein said other chemotherapeutic agent is administered simultaneously with said N⁶-benzyladenosine, N⁶-benzyladenosine-5′-monophosphate, or pharmaceutically acceptable salt thereof.

12. The method according to claim 11, wherein said other chemotherapeutic agent and said N⁶-benzyladenosine, N⁶-benzyladenosine-5′-monophosphate, or pharmaceutically acceptable salt thereof, are administered in the same dosage form.

13. The method according to claim 9, wherein said other chemotherapeutic agent is selected from the group consisting of dasatinib, nilotinib, nelarabine, vincristine, daunorubicine, idarubicine, cytarabine, L-asparaginase, etoposide, teniposide, 6-mercaptopurine, methotrexate, cyclophosphamide, prednisone, 6-thioguanine, hydroxyurea, ATRA, ATO, fludarabine, pentostatin, cladribine, chlorambucil, bendamustine, oxaliplatin, etoposide, topotecan, azacytidine, decitabine, rituximab, alemtuzumab and ofatumumab.
